(19) United States
(12) Patent Application Publication
(10) Pub. No.: US 2010/0055204 A1
(43) Pub. Date: Mar. 4, 2010

(54) USE OF HYDROCHLORIC ACID IN THE MANUFACTURE OF MEDICAMENT FOR TREATING DISEASE CAUSED BY FOULING OF TRANSPORT TRACT

(76) Inventor: Zhiren Liu, Zhejiang (CN)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/515,785

(22) PCT Filed: Sep. 7, 2007
(86) PCT No.: PCT/CN2007/002668

§ 371 Date: May 21, 2009

(30) Foreign Application Priority Data
Sep. 8, 2006 (CN) 200610053309.2

(51) Int. Cl. A61K 33/00; A61P 3/10

(52) U.S. Cl. 424/666

(57) ABSTRACT

Use of hydrochloric acid in the manufacture of a medicament for treating the disease caused by fouling of transport tract, wherein the disease comprises diabetes and prostate disease.

### FIELD OF THE INVENTION

The present invention relates to a medicament using hydrochloric acid for treating human organ diseases caused by fouling of human organ and of transport tract in human body, and particularly relates to use of dilute hydrochloric acid in a medicament for treating the diseases including diabetes and prostate diseases caused by fouling of human organ and of the inner wall of cardiac blood vessel.

### BACKGROUND OF THE INVENTION

The cause and pathogenesis of diabetes are very complicated, and are not fully understood so far, which according to literature disclosed by IDF (International Diabetes Federation) and WHO (World Health Organization) in 1999. But, at present, traditional theory and modern medicine both believe that blood glucose level is closely related to the secretion of insulin and the amount and species of diets. Medical professions also believe that diabetes has no radical cure by far, and can only be controlled by following measures, i.e. diet control, oral administration of hypoglycemic agents, injection administration of insulin, or oral administration of hypoglycemic agents in combination with injection administration of insulin.

Similarly, the cause and pathogenesis of prostate diseases, particularly chronic prostate diseases, are not well understood so far. It is generally believed that acute prostate diseases are mainly caused by bacteria, while etiology of chronic prostate diseases is very complicated; although various pathogenesis of chronic prostate diseases are learned to some extent, no breakthrough is attained, and no radical cure is identified either. At present, it is considered that chronic prostate diseases could be protopathic or secondary diseases of prostate and its peripheral tissue organs, muscles, and nerves; therefore antibiotic treatment or local treatment are frequently adopted, such as local drug administration, massage of prostate, hot hip bath, local physical therapy, and biofeedback technique.

### DISCLOSURE OF THE INVENTION

### Problem to be Solved by the Present Invention

At present, blood glucose control is the main target for treating the above mentioned diabetes, either lowering the intake of glucose, or artificially increasing of insulin level is adopted to make glucose smoothly decomposed. But no matter what method is adopted, lifelong drug administration or insulin injection and diet control are indispensable. In addition, those treatment methods can only be effective as to control the deterioration rate of diabetes and its complications, however, the disease will be getting worse along with aging.

Similarly, prostate diseases are also considered to be caused by various factors; therefore, optimized combination method is adopted, i.e. antibiotic treatment in combination with other treatment methods. But in long term either systematic, or local antibiotic administration will bring severe damage to human immune system and nervous system; however,other physical therapeutic methods require long term treatment process, and certain auxiliary equipments, moreover, impose certain restrictions on patients' daily life, and increase economic burden, thus lowering patients' life quality, particularly, causing great psychological pain due to recurrence of the symptoms.

For solving the aforementioned issues, based on research and practice in many years, the inventor of the present invention has found that the formation of diabetes and prostate diseases is based on the following mechanism, i.e. adhesives calcium carbonate and calcium oxalate, which are generated from calcium bicarbonate in water and food via decomposition, adhere human body metabolites (such as cholesterol crystal, etc.) and solid matter in human body to human organ and the inner wall of cardiac blood vessel used for transporting various components and metabolites, which prevents transporting, even causes blockage of transportation path, further leads to inflammation, and finally results in the formation of diabetes and prostate diseases.

From the above mechanism, it can be learned that the cause of diabetes is that:
1. Adhesives calcium carbonate and calcium oxalate, which are generated from calcium bicarbonate in water and food via decomposition, adhere human body metabolites (such as cholesterol crystal, etc.) and solid matter in human body to the inner wall of capillary blood vessel used for transporting blood glucose and to the cell entrance used for blood glucose to enter into cell, and thus form fouling (calcification) in capillary blood vessel and cell entrance. With aging, the fouling on the inner wall of capillary blood vessel and the cell entrance gets thicker, and the fouling area gets larger, both of which prevent a large amount of blood glucose and other nutritional substance from entering into and being consumed by cells; therefore, a large amount of blood glucose and other nutritional substance retain in blood to cause diabetes. This type of diabetes has been known as Type II diabetes.
2. Adhesives calcium carbonate and calcium oxalate, which are generated from calcium bicarbonate in water and food via decomposition, adhere human body metabolites (such as cholesterol crystal, etc.) and solid matter in human body to pancreas and to the tract wall used for transporting insulin. When the fouling accumulates to certain extent, following three conditions may occur:
   (1) A large amount of fouling accumulates in pancreas, however the tract used for transporting insulin is unblocked; due to the fouling in pancreas, this negatively affects insulin secretion amount, and the secreted insulin activity is poor.
   (2) Pancreas is intact, however, the insulin transporting tract accumulates fouling, and thus insulin secreted by pancreas is contaminated when passing through the blocked transportation tract, and therefore, weakened the capability for transporting blood glucose.
   (3) Pancreas and insulin transportation tract both accumulate fouling, the amount of insulin transported to blood is less, and the insulin activity is poor.

Insulin is a substance for carrying blood glucose while entering into cell for being consumed; due to the deficiency of carrying vehicle and its poor activity, any one of the above problems will prevent a large amount of blood glucose which should be absorbed and consumed by cells, from entering into cells for being absorbed and consumed, and thus leads to blood glucose elevation and over nutrition in the blood. When blood glucose is excess the normal value, this will results in diabetes. This type of diabetes has been known as Type I diabetes.

Based on the above analysis, the inventor of the present invention deems that the present treatment measures can only alleviate symptoms or delay deterioration rate of diabetes, but can not radically cure the disease.

Moreover, from the above mechanism, it can be learned that the cause of prostate diseases is because the calcium carbonate and calcium oxalate adhere metabolites to the prostate, following of which results in the diseases, ie, hyperplasia and calculus of prostate, etc.

Based on the above analysis, the inventor of the present invention deems that this is the reason why the damage and pathological change caused by prostate diseases still independently grow even when the diseases of prostate and its peripheral tissue organs, muscles, and nerves are cured or thoroughly eradicated during treatment by medical professions. Even when the inflammation caused by bacteria is eliminated, a certain kind of physical state change is still not eliminated, and thus the disease will relapse after “cured” for a certain time. That's also the reason why chronic prostate diseases have very high incidence rate (45-25%), and almost 50% males will encounter the symptoms of prostatitis in certain moment of their life, particularly in adult males. Therefore, no matter how big the difference of the life style and diet habit in each individual can be, metabolites with adhesive effects, such as calcium carbonate and calcium oxalate, will exist in metabolic substance of human body ultimately. Along with aging, the aforementioned adhesives casue the fouling of prostate accumulate and thus lead to prostate diseases; and also cause the fouling of bladder increase and thus lead to reduction of bladder volume and its contractibility, consequently result in frequent micturition, and incomplete urination.

From explanation of above symptoms, the inventor has found and deemed that of all the organs and tracts with transportation function in human body will generate fouling, once fouling accumulates, a series of diseases will arise, for example, acquired obstruction of spermaduct and oviduct, also calculus, onychomycosis, gout, senile plaque, breast lobule hyperplasia, dull skin, gynaecopathia, hemorrhoids and hyperostosis, etc. The functions can get back to normal as long as fouling on the transportation duct wall, inner wall of secretory gland, cell entrance, and capillary blood vessel wall is removed. To remove the fouling, adhesives calcium carbonate and calcium oxalate must be dissolved, and then other attached small particulate matters can be separated and excreted out of the body.

In other words, the present invention aims to solve the above problems, and to provide a substance applied in medicament for treating human organ diseases caused by fouling of human organs and of transportation tract in human body.

The object of the present invention further comprises providing a substance applied in medicament for treating diseases including diabetes caused by fouling of the inner wall of cardiac blood vessels.

Another object of the present invention is to provide a substance applied in preparation of medicament for treating diabetes.

Another object of the present invention is to provide a substance applied in medicament for treating diseases including prostate diseases caused by fouling of the inner wall of organs in human body.

### Method for Solving the Problems

Based on the aforementioned mechanism, after research in depth, the inventor has found that hydrochloric acid is the most ideal substance for dissolving calcium carbonate and calcium oxalate in human body, at the same time, hydrochloric acid can be adsorbed by the protection membrane on inner wall of the cells, and repair damaged blood vessels and pancreas. Additionally, the inventor has found that hydrochloric acid is beneficial for dissolving of hyperplasia and calculus of the prostate, and thus makes prostate recover from the diseases.

Therefore, for solving the above issues, the present invention provides a medicament using hydrochloric acid for treating human organ diseases caused by fouling of human organs and of transport tract in human body.

The present invention further provides a medicament using hydrochloric acid for treating the diseases including diabetes caused by fouling of the inner wall of cardiac blood vessels.

Moreover, the present invention provides a medicament using hydrochloric acid for treating diabetes.

The present invention further provides a medicament using hydrochloric acid for treating diseases including prostate diseases caused by fouling of the inner wall of human organs.

Moreover, the present invention provides a medicament using hydrochloric acid for treating prostate diseases.

Additionally, the volume concentration of hydrochloric acid in the present invention is preferably 0.005%-0.1%.

The pH of the aqueous solution of hydrochloric acid in the present invention is preferably controlled within 1-6.9.

### Effect of the Present Invention

For explaining therapeutic effect of hydrochloric acid to diabetes in the present invention, 106 diabetes patients who are oral administration hydrochloric acid aqueous solution, are observed clinically, in which the number of male patients is 56, and the number of female patients is 50, in addition, the average age is 59, and the age range is 46-72. The practice result shows that the number of the diabetes patients using edible hydrochloric acid aqueous solution in combination with hypoglycemic agents, and the diabetes patients only using edible hydrochloric acid aqueous solution who are completely cured, is 45; the number of the diabetes patients using edible hydrochloric acid aqueous solution in combination with hypoglycemic agents, whose blood glucose index has been controlled within normal range and have reduced administration of hypoglycemic agents, is 31; the number of diabetes patients, whose blood glucose index unable to be controlled within normal range by oral administration of hypoglycemic agents and by injection insulin, after oral administration of edible hydrochloric acid aqueous solution for two years, have blood glucose index controlled within normal range by using same oral hypoglycemic agents and insulin injection, is 30.

Additionally, the present invention has carried out clinical observation of prostate hyperplasia and calculus patients taking edible hydrochloric acid solution; the result shows that oral administration of hydrochloric acid solution is helpful for alleviating and eliminating prostate hyperplasia and calculus.

The medicament prepared from hydrochloric acid in the present invention is safe and reliable for treating diabetes and prostate diseases, and has exact and significant effect, moreover capable of converting calcium salt harmful for human body into calcium benefit for human body.

### Embodiment

The present invention will be further explained through following embodiments. It should be understood that those embodiments are only used for explaining the present invention while not for limiting scope of the present invention, and other implementation manners included in the claim scope are also applicable.

In the following embodiments, traditional conditions or conditions recommended by manufacturers are adopted in the experiment methods unless specified otherwise.

### Embodiment 1

2% of high purity hydrochloric acid is added into drinking water, stirred well, regulated to pH of 1-6.9, and diluted to volume concentration (V/V) of 0.005%-0.1%; to be specific, the pH value can be regulated to 1, 6.9, 3, 6.5, 6.7, 4.5, or 2.0, and the volume concentration after dilution can be 0.005%, 0.1%, 0.015% or 0.01%. The prepared hydrochloric acid-containing water solution can be directly taken as edible hydrochloric acid solution. Subjects to be treated are administered with the hydrochloric acid solution at 100-3,000 ml per day on average, preferably not less than 1,500 ml by taking separately 3-6 times per day. 3-6 months is one treatment course, which will last about 0.5-6 years.

When tap water is adopted for preparing hydrochloric acid solution, purification device should be added at front end to make the tap water satisfy national drinking water standard.

The temperature of the edible hydrochloric acid solution in the present invention is preferably room temperature; basically heating is not allowed, for heating will volatilize hydrochloric acid and lost its efficacy. Beverage and tea incline to fouling are not recommended, for theme will neutralize hydrochloric acid and lost its efficacy.

### Embodiment 2

0.005% (V/V) hydrochloric acid aqueous solution with pH 6.9 is prepared into injection. When injected for treatment of diabetes, the recommended dosage is 500 ml per day injected by two times in intravenous drip manner, and 2-3 months are one treatment course. By treating diabetes according to the above method and dosage, the dosage for oral administration of hypoglycemic agents and injection of insulin can be reduced by ¼ after one year, reduced by half after two years, and reduced to maintenance dose or dose free after three years, and thus the blood glucose can be back to normal.

### Embodiment 3

High purity hydrochloric acid is added into drinking water to be diluted into 2% hydrochloric acid solution, and filled in container with flow rate control. The hydrochloric acid solution and drinking water are mixed at certain flow rate ratio via pipes, and pH detector is provided at the end of the pipes. pH of the hydrochloric acid solution is controlled within 4-6.9 to give 0.5% (V/V) edible hydrochloric acid solution.

15 diabetes patients administered with the above edible hydrochloric acid solution are clinically observed. The solution is expected administered at 2,000 ml per day, in which 500 ml is respectively administered one hour before breakfast and lunch, and 1,000 ml is administered one hour before supper.

Table 1 shows the improvement of diabetes condition of partial patients after oral administration of the hydrochloric acid solution in the present invention.

TABLE 1
treating diabetes with edible hydrochloric acid solution

| Patient | Fasting blood glucose | | Treatment effect of diabetes (in which the |
|---|---|---|---|
| No. | (mmmol/L) | Age | number is fasting blood glucose value) |
| 1 | 7.1 | 50 | respectively reduced to 6.7 and 6.1 after |
| | | | administration of hydrochloric acid |
| | | | solution for half year and one year |
| 2 | 7.3 | 52 | respectively reduced to 6.6 and 5.9 after |
| | | | administration of hydrochloric acid |
| | | | solution for one year and two years |
| 3 | 7.2 | 61 | respectively reduced to 6.8 and 6.2 after |
| | | | administration of hydrochloric acid |
| | | | solution for one year and two years |
| 4 | 7.1 | 69 | respectively reduced to 6.8 and 6.5 after |
| | | | administration of hydrochloric acid |
| | | | solution for one year and two years |
| 5 | 7.2 | 71 | respectively reduced to 7 and 6.6 after |
| | | | administration of hydrochloric acid |
| | | | solution for one year and two years |
| 6 | 6.1 (oral administration of | 46 | after administration of hydrochloric acid |
| | hypoglycemic agent) | | solution for half year, hypolycemic agent |
| | | | dosage is reduced by ½; after |
| | | | administration of hydrochloric acid |
| | | | solution for two years, administration of |
| | | | hypoglycemic agent is stopped, and |
| | | | fasting blood glucose value is reduced to |
| | | | 6.0. |
| 7 | 6.5 (oral administration of | 48 | after administration of hydrochloric acid |
| | hypoglycemic agent) | | solution for one year, hypolycemic agent |
| | | | dosage is reduced by ½; after |
| | | | administration of hydrochloric acid |
| | | | solution for two years, administration of |
| | | | hypoglycemic agent is stopped, and |
| | | | fasting blood glucose value is reduced to |
| | | | 6.1. |
| 8 | 6.7 (oral administration of | 53 | after administration of hydrochloric acid |
| | hypoglycemic agent) | | solution for one year, hypolycemic agent |
| | | | dosage is reduced by ⅓; after |
| | | | administration of hydrochloric acid |
| | | | solution for two years, administration of |
| | | | hypoglycemic agent is reduced by half; |
| | | | after administration of hydrochloric acid |
| | | | solution for three years, administration of |
| | | | hypoglycemic agent is stopped, and |
| | | | fasting blood glucose value is reduced to |
| | | | 6.2. |
| 9 | 6.8 (injection | 66 | after administration of hydrochloric acid |
| | administration of insulin) | | solution for one year, insulin injection |
| | | | dosage is reduced by ½; after |
| | | | administration of hydrochloric acid |
| | | | solution for two years, insulin injection is |
| | | | stopped, and fasting blood glucose value |
| | | | is reduced to 6.3 |
| 10 | 6.0 (oral administration of | 59 | after administration of hydrochloric acid |
| | hypoglycemic agent, and | | solution for one year, oral administration |
| | injection administration of | | of hypoglycemic agent is stopped; after |
| | insulin) | | administration of hydrochloric acid |
| | | | solution for two years, insulin injection |
| | | | dosage is reduced by half, and fasting |
| | | | blood glucose value is reduced to 5.9. |
| 11 | 7.9 (oral administration of | 52 | Fasting blood glucose value is reduced to |
| | hypoglycemic agent) | | 6.9 after administration of hydrochloric |
| | | | acid solution for one year, the |
| | | | hypoglycemic agent dosage is reduced by |
| | | | half after administration of hydrochloric |
| | | | acid solution for 2 years, oral |
| | | | administration of hypoglycemic agent is |
| | | | stopped after administration of |
| | | | hydrochloric acid for three years, and |
| | | | fasting blood glucose value is reduced to |
| | | | 6.3. |
| 12 | 7.8 (oral administration of | 72 | Fasting blood glucose values are |
| | hypoglycemic agent) | | respectively reduced to 7.1 and 6.6 after |
| | | | administration of hydrochloric acid |
| | | | solution for one year and two years, the |
| | | | hypoglycemic agent dosage is reduced by |
| | | | half after administration of hydrochloric |
| | | | acid solution for 3 years, and fasting blood |
| | | | glucose value is reduced to 6.2. |
| 13 | 11 (oral administration of | 51 | Fasting blood glucose values are |
| | hypoglycemic agent) | | respectively reduced to 7.6 and 6.5 after |
| | | | administration of hydrochloric acid |
| | | | solution for one year and two years, the |
| | | | hypoglycemic agent dosage is reduced by |
| | | | half after administration of hydrochloric |
| | | | acid solution for 3 years, and fasting blood |
| | | | glucose value is reduced to 6.1. |
| 14 | 9.8 (oral administration of | 66 | after administration of hydrochloric acid |
| | hypoglycemic agent, and | | solution for 1 year, oral administration of |
| | injection administration of | | hypoglycemic agent is stopped, insulin |
| | insulin) | | injection dosage is kept the same, and |
| | | | fasting blood glucose value is reduced to |
| | | | 7.6; after administration of hydrochloric |
| | | | acid solution for 2 years, insulin injection |
| | | | dosage is reduced by ⅓, and fasting |
| | | | blood glucose value is reduced to 7. |
| 15 | 13 (oral administration of | 69 | Fasting blood glucose values are |
| | hypoglycemic agent, and | | respectively reduced to 9.5 and 7.8 after |
| | injection administration of | | administration of hydrochloric acid |
| | insulin) | | solution for one year and two years; after |
| | | | administration of hydrochloric acid |
| | | | solution for 3 years, the hypoglycemic |
| | | | agent dosage is reduced by half, and |
| | | | fasting blood glucose value is reduced to |
| | | | 6.5. |

Generally, administration dosages of oral hypoglycemic agent and/or insulin injection for diabetes patients are increased along with time. While it can be observed that after administration of edible hydrochloric acid solution in the present invention, the administration dosages of oral hypoglycemic agent and/or insulin injection can be decreased along with time, and the fasting blood glucose value can be effective controlled and reduced at the same time. The hydrochloric acid solution has significant effective for treating diabetes, with total effective rate of 100%.

### Embodiment 4

High purity hydrochloric acid is added into drinking water to be diluted into 2% hydrochloric acid solution, and filled in container with flow rate control The hydrochloric acid solution and drinking water are mixed at certain flow rate ratio via pipes, and pH detector is provided at the end of the pipes. pH of the hydrochloric acid solution is controlled within 4.0-6.5 to give 0.015% (V/V) edible hydrochloric acid solution.

Multiple prostate diseases patients administered with the above edible hydrochloric acid solution are clinically observed. According to the above hydrochloric acid solution with pH of 6.5, the solution is administered at 2,000 ml per day, in which 500 ml is respectively administered one hour before breakfast and lunch, and 1,000 ml is administered one hour before supper. According to the above hydrochloric acid solution with pH of 5.0, the solution is administered at 1,000 ml per day, in which 250 ml is respectively administered one hour before breakfast and lunch, and 500 ml is administered one hour before supper.

The treatment result is shown in Table 2.

TABLE 2
Treating hyperplasia and calculus of prostate with edible hydrochloric acid solution

| | Symptom of hyperplasia of prostate, | | Treatment effect of hyperplasia and |
|---|---|---|---|
| Name | calculus diameter (cm) | Age | calculus of prostate |
| Zhang X | 0.3, urine splitting | 51 | calculus disappears after administration of |
| | | | the hydrochloric acid solution for half |
| | | | year, and the urine is back to normal after |
| | | | administration of the hydrochloric acid |
| | | | solution for one year. |
| Wang X | difficulty of urination, | 53 | the dosage of oral preparation is reduced |
| | feel of incomplete | | by half after administration of the |
| | urination | | hydrochloric acid solution for half year; |
| | | | after administration of the solution for 1.5 |
| | | | years, administration of the oral |
| | | | preparation is stopped, and the urination is |
| | | | back to normal. |
| Jiang X | frequent micturition, | 61 | the dosage of oral preparation is reduced |
| | urgent micturition | | by half after administration of the |
| | | | hydrochloric acid solution for one year; |
| | | | after administration of the solution for 2 |
| | | | years, administration of the oral |
| | | | preparation is stopped, and the urination is |
| | | | back to normal. |
| Huang X | 0.2, difficulty of | 68 | calculus disappears after administration of |
| | urination, urination | | the hydrochloric acid solution for one |
| | interruption | | year, dosage of oral preparation is reduced |
| | | | by half after administration of the solution |
| | | | for two years, and urination interruption |
| | | | disappears. |
| Liu X | difficulty of urination, | 73 | the dosage of oral preparation is reduced |
| | frequent micturition at | | by ⅓ after administration of the |
| | night | | hydrochloric acid solution for one year; |
| | | | after administration of the solution for 2 |
| | | | years, the dosage of the oral preparation is |
| | | | reduced by half, and the urination is back |
| | | | to normal. |
| Li X | incomplete urination, 0.4 | 59 | calculus is reduced to 0.2 after |
| | | | administration of the solution for one year; |
| | | | after administration of the solution for two |
| | | | years, calculus disappears, the dosage of |
| | | | the oral preparation is reduced by half, and |
| | | | incomplete urination disappears. |
| Zhao X | difficulty of urination, | 67 | the dosage of oral preparation is reduced |
| | frequent micturition at | | by half after administration of the |
| | night | | hydrochloric acid solution for one year; |
| | | | after administration of the solution for 2 |
| | | | years, the administration of the oral |
| | | | preparation is stopped, and the urination is |
| | | | back to normal. |
| Yuan X | difficult to hold urine, | 66 | the dosage of oral preparation is reduced |
| | incomplete urination, | | by half after administration of the |
| | and frequent micturition | | hydrochloric acid solution for two years; |
| | | | after administration of the solution for 3 |
| | | | years, the administration of the oral |
| | | | preparation is stopped, and the urination is |
| | | | back to normal. |
| Xu X | small urine amount, | 73 | after administration of the solution for one |
| | frequent micturition, | | year, the dosage of oral preparation is |
| | incomplete urination, | | reduced by half, and difficulty of urination |
| | and difficulty of | | disappears; after administration of the |
| | urination | | solution for three years, the dosage of the |
| | | | oral preparation is reduced by half, and |
| | | | urination is back to normal. |
| Lin X | frequent micturition, | 49 | urination back to normal after |
| | urgent micturition, small | | administration of the solution for one year |
| | urine amount | | |

It can be observed that the hydrochloric acid solution has significant effective in treating hyperplasia and calculus of prostate.

### Embodiment 5 Compound Preparation of Hydrochloric Acid Solution+diabetes Medicinal Preparation

One tablet of commercially available Tolbutamide (2.5 mg) is added into 500 ml hydrochloric acid solution with pH 6.9, to constitute compound preparation containing hydrochloric acid. Treatment effects similar to No. 6-8 in Table 1 can be obtained when the compound preparation is applied for treating diabetes.

Similarly, other compound preparations of hydrochloric acid+other diabetes medicament can be prepared.

The aforementioned embodiments only aim at partial human diseases caused by transport tract fouling in human organs in the present invention; after the contents of the present invention are learned, it can be understood that the present invention is applicable to treatment of all diseases caused by aforementioned causes. For example, one cause of onychomycosis (tinea unguium) is related to diseases of local artery and vein circulation and lymphatic return difficulty, if medicament prepared from the diluted hydrochloric acid solution in the present invention is adopted to improve or treat local artery and vein circulation and lymphatic return difficulty, treatment effect can be speeded up or consolidated. Alternatively, one cause of osteoporosis and hyperostosis is that capillary blood vessel at bone part, particularly joint part, is obstructed or blocked which causes nutrients in blood unable to be supplied to bone, therefore, bone can only consume its own nutrients, then calcium compounds are retained, as calcium compounds will expand, symptoms of osteoporosis and hyperostosis are arise; if the medicament prepared from the diluted hydrochloric acid solution in the present invention is adopted to improve circulation of capillary blood vessel, the symptoms can be eliminated. Additionally, one cause of heart diseases is also fouling of the heart and of the cardiac blood vessels, which leads to angina pectoris, bearing premature, coronary artery disease, and heart failure, when fouling of the heart and of the cardiac blood vessels falls and blocks coronary artery and its branches, myocardial infarction is formed; if the medicament prepared from the diluted hydrochloric acid solution in the present invention is adopted to improve circulation of cardiac blood vessels, the symptoms can be eliminated. Moreover, one cause of breast lobule hyperplasia is that when local capillary blood vessels and artery and vein of breast are blocked to cause local tissues basification and thus arise lobule hyperplasia; if the medicament prepared from the diluted hydrochloric acid solution in the present invention is adopted to improve circulation of cardiac blood vessels, artery and vein, the symptoms can be eliminated. Also, one cause of gout is fouling of capillary blood vessels at joint part, nutrients can not be supplied to joint bone via capillary blood vessels, therefore, nutrients of the bone are absorbed and consumed by bone itself, the retained calcium compounds expand, the swollen bone compresses artery and vein passing through the bone, to deteriorate dilation and contraction of the blood vessels and further speed up fouling of the blood vessels, the diameter of the blood vessel becomes smaller, gout is formed when urea crystals generated from reaction of alkaline matters in blood and uric acid in blood pass this part; if the medicament prepared from the diluted hydrochloric acid solution in the present invention is adopted to improve circulation of capillary blood vessels, and to inhibit the bone swelling and calcification, then, the disease can be prevented, while, for patients already has the disease, fouling of the artery and vein blood vessel near the bone can be improved or eliminated, and thus the symptoms can be alleviated or eliminated. What's more, one cause of haemorrhoids is fouling of artery and vein at periphery of anus, which causes obstruction of the blood vessel at this part, pressure at this part is increased due to intestines peristalsis during defaecation, blood is forced to pass the obstructed part, which leads to rupture and incrustation of blood vessel, and results in hardening, deformation, and bulging out of the blood vessel to form haemorrhoids; if the medicament prepared from the diluted hydrochloric acid solution in the present invention is adopted to improve circulation of cardiac blood vessel, artery and vein, the symptoms can be eliminated. Likewise, one cause of male sexual dysfunction or impotentia is fouling of blood vessel in penis; if the medicament prepared from the diluted hydrochloric acid solution in the present invention is adopted to improve circulation of blood vessel, the symptoms can be eliminated. Additionally, one cause of gynecological disease like irregular menstruation, vaginitis, and salpingitis is fouling of transport tract of the female reproductive organ, which leads to obstruction and inflammation; if the medicament prepared from the diluted hydrochloric acid solution in the present invention is adopted to improve circulation of the transport tract, the symptom can be eliminated.

Furthermore, it should be understood that those skilled in arts can modify or revise the present invention in various manners after the contents of the present invention are read. For example, the aqueous solution can be prepared from common drink water, such as physiologic saline, glucose solution, or juice etc; the preparation method is the same as aforementioned method, as long as pH of the oral liquid or injection is controlled. In other words, any aqueous solution, won't react with hydrochloric acid to create chemical substances unbeneficial to human body, is applicable, and their equivalent falls into the scope of the present invention as well.

What is claimed is:

1. Use of hydrochloric acid as a medicament for treating human organ diseases caused by fouling of human organ and of transport tract in human body.

2. The use of hydrochloric acid as a medicament for treating diseases caused by fouling of human organ and of transport tract in human body according to claim 1, which is characterized in that the medicament is hydrochloric acid used for treating diseases including diabetes caused by fouling of the inner wall of cardiac blood vessel.

3. The use of hydrochloric acid as a medicament for treating diseases caused by fouling of human organ and of transport tract in human body according to claim 2, which is characterized in that the medicament is hydrochloric acid used for treating diabetes.

4. The use of hydrochloric acid as a medicament for treating diseases caused by fouling of human organ and of transport tract in human body according to claim 1, which is characterized in that the medicament is hydrochloric acid used for treating diseases including prostate diseases caused by fouling of the inner wall of human organs.

5. The use of hydrochloric acid as a medicament for treating diseases caused by fouling of human organ and of transport tract in human body according to claim 4, which is characterized in that the medicament is hydrochloric acid used for treating prostate diseases.

6. The use of hydrochloric acid as a medicament for treating diseases caused by fouling of human organ and of transport tract in human body according to claim 1, which is characterized in that the volume concentration (V/V) of the aqueous solution of hydrochloric acid is 0.005%-0.1%.

7. (canceled)

8. The use of hydrochloric acid as a medicament for treating diseases caused by fouling of human organ and of transport tract in human body according to claim 2, which is characterized in that the volume concentration (V/V) of the aqueous solution of hydrochloric acid is 0.005%-0.1%.

9. The use of hydrochloric acid as a medicament for treating diseases caused by fouling of human organ and of transport tract in human body according to claim 3, which is characterized in that the volume concentration (V/V) of the aqueous solution of hydrochloric acid is 0.005%-0.1%.

10. The use of hydrochloric acid as a medicament for treating diseases caused by fouling of human organ and of transport tract in human body according to claim 4, which is characterized in that the volume concentration (V/V) of the aqueous solution of hydrochloric acid is 0.005%-0.1%.

11. The use of hydrochloric acid as a medicament for treating diseases caused by fouling of human organ and of transport tract in human body according to claim 5, which is characterized in that the volume concentration (V/V) of the aqueous solution of hydrochloric acid is 0.005%-0.1%.

12. The use of hydrochloric acid as a medicament for treating diseases caused by fouling of human organ and of transport tract in human body according to claim 6, which is characterized in that the pH of the aqueous solution of hydrochloric acid is controlled within 1-6.9.

13. The use of hydrochloric acid as a medicament for treating diseases caused by fouling of human organ and of transport tract in human body according to claim 7, which is characterized in that the pH of the aqueous solution of hydrochloric acid is controlled within 1-6.9.

14. The use of hydrochloric acid as a medicament for treating diseases caused by fouling of human organ and of transport tract in human body according to claim 8, which is characterized in that the pH of the aqueous solution of hydrochloric acid is controlled within 1-6.9.

15. The use of hydrochloric acid as a medicament for treating diseases caused by fouling of human organ and of transport tract in human body according to claim 9, which is characterized in that the pH of the aqueous solution of hydrochloric acid is controlled within 1-6.9.

16. The use of hydrochloric acid as a medicament for treating diseases caused by fouling of human organ and of transport tract in human body according to claim 10, which is characterized in that the pH of the aqueous solution of hydrochloric acid is controlled within 1-6.9.
